(19) United States
(12) Patent Application Publication
(10) Pub. No.: US 2018/0000637 A1
(43) Pub. Date: Jan. 4, 2018

(54) PORTABLE MOXIBUSTION THERAPY DEVICE

(71) Applicant: YONGWOO JUNG, COSTA MESA, CA (US)

(72) Inventor: YONGWOO JUNG, COSTA MESA, CA (US)

(21) Appl. No.: 15/199,050

(22) Filed: Jun. 30, 2016

(51) Int. Cl. A61F 7/03; A61F 7/00; A61F 7/02

(52) U.S. Cl. CPC A61F 7/032; A61F 2007/0292; A61F 2007/0228; A61F 2007/0027; A61F 2007/0042; A61F 2007/003; A61F 2007/0204

(57) ABSTRACT

A portable moxibustion therapy device includes a main body 20, having a top opening 21, a sidewall 22 and a bottom 23, and the sidewall 22 comprises a plurality of holes 24 on the sidewall 22 of the main body 20; a cover 30 for opening and closing the top opening 21 of the main body 20; a moxa container 40 which is substantially cylindrical and made of yellow soil in the main body 20; a cylindrical support 50 made of yellow soil placed the moxa container 40 blow in the main body 20; and a gauze 60 located between the moxa container 40 and the cylindrical support 50 in the main body 20. The portable moxibustion therapy device includes a plurality of holes to supply air or oxygen to the moxa 90.

### CROSS-REFERENCE TO RELATED APPLICATIONS

Not applicable.

### FIELD OF THE INVENTION

The present invention relates to a portable moxibustion therapy device for relieving pain on a user's and, more particularly, to the portable moxibustion therapy device having a moxa container to receive a moxa therein made of dried mugwort and a cylindrical support to support the moxa. The portable moxibustion therapy device is easy to carry and very convenient to use by placing it on a user's skin.

### BACKGROUND OF THE INVENTION

Moxibustion or moxa therapy, is a traditional Asian medical treatment therapy to relieve or treat a pain or stiff muscles of a person such as pain on back, knees or shoulders. Moxibustion uses heat from a burning moxa so that the heat is transferred to a person's skin and the moxa is made of mugwort.

There are two types of moxibustion—direct and indirect—depending on the way of burning moxa. In direct combustion, moxa is placed to form a small cone shape and lit. Then, the heat from the burning moxa is transferred to an acupuncture point of a person. However, direct moxibustion may lead to a partial pain, burn, scar or blister after the therapy.

Thus, indirect moxibustion has become more popular. It uses a moxa stick which is lit and placed closed to a person's skin to transfer heat from the burning moxa to a person's skin. However, a practitioner has to hold the burning moxa stick close to a patient's skin for an extended period of time and thus, indirect moxibustion is inconvenient to practice.

Besides, during either way of moxa combustion, a patient should stay still and should not move because of the burning moxa placed close to the patient's skin.

Therefore, to solve the above problems, there is a need for a portable convenient-to-use free-of-burn moxibustion therapy device for relieving pain. This invention is directed to solve these problems and satisfy the long-felt need.

### SUMMARY OF THE INVENTION

The present invention contrives to solve the disadvantages of the prior art. The present invention provides a portable moxibustion therapy device for pain of a body of user and, more particularly, to the portable moxibustion therapy device having a moxa container to contain a burning moxa made of dried mugwort and therein a cylindrical support to support the moxa. The portable moxibustion therapy device can be carry out with attaching the body of user and is very convenient to use. The object of the present invention is to provide a portable moxibustion therapy device for treating pain of a body of user using a burning moxa made from dried mugwort, the portable moxibustion therapy device which includes a main body, having a top opening, a sidewall and a bottom; a cover for opening and closing the top opening of the main body; a moxa container which is substantially cylindrical and made of yellow soil in the main body; a cylindrical support made of yellow soil placed the moxa container blow in the main body; and a gauze located between the moxa container and the cylindrical support in the main body. The sidewall includes a plurality of holes on the sidewall of the main body.

Another object of the present invention is to provide a potable moxibustion therapy device having a main body and a support. The device includes a cover for opening and closing the top opening of the main body; a moxa container which is substantially cylindrical and made of yellow soil in the main body; a cylindrical support made of yellow soil placed the moxa container blow in the main body; a plurality of balls made of mineral material or yellow soil on the bottom of the main body; a first gauze located between the moxa container and the cylindrical support in the main body; and a second gauze covering the cylindrical support and the plurality of balls.

The advantages of the present invention are: (1) the device of the present invention is constructed to prevent burn, scar, or blister on a person's skin from the moxibustion therapy; (2) the device of the present invention is effective in transferring heat from burning moxa to a person's skin; (3) the device of the present invention is easy and convenient to use and a user can move around while getting moxibustion treatment from the device; (4) the moxa container, cylindrical supporter, and bottom of the main body form a heat passage for effectively transferring heat from the burning moxa to a person's skin and the heat is transferred to a broader area of a person's skin for better treatment; (5) during the moxibustion treatment, the device emits a lot of far infrared heat which is transferred to a person's skin; and (6) in the device, the moxa container, the supporter and the plurality of balls are made of yellow soil such that they emit and release far infrared and negative ion for treating and stimulating a person along with the moxibustion.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIGS. 1A and 1B show front perspective views of a portable moxibustion therapy device according to one of embodiment of the present invention;

FIG. 2 shows another perspective view of the portable moxibustion therapy device according to the present invention when a cover is opened along with moxa;

FIG. 3 shows a front exploded view of the portable moxibustion therapy device according to the present invention;

FIG. 4 shows a cross sectional view of the portable moxibustion therapy device along 4-4 line of FIG. 6;

FIG. 5 shows another perspective view of the portable moxibustion therapy device according to the present invention wrapped or covered with fabric;

FIG. 6 shows a top plan view of the portable moxibustion therapy device according to the present invention;

FIG. 7 shows a top plan view of the portable moxibustion therapy device according to the present invention when the cover is opened;

FIG. 8 shows a bottom plan view of the portable moxibustion therapy device according to the present invention;

FIGS. 9A, 9B and 9C show front elevation views of the portable moxibustion therapy device according to the present invention attached the body of user respectively such as back, shoulders and knees.

### DETAILED DESCRIPTION EMBODIMENTS OF THE INVENTION

Reference will now be made in detail to the preferred embodiments of the present invention, examples of which are illustrated in the accompanying drawings, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention.

Also, as used in the specification including the appended claims, the singular forms “a”, “an”, and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about”, it will be understood that the particular value forms another embodiment.

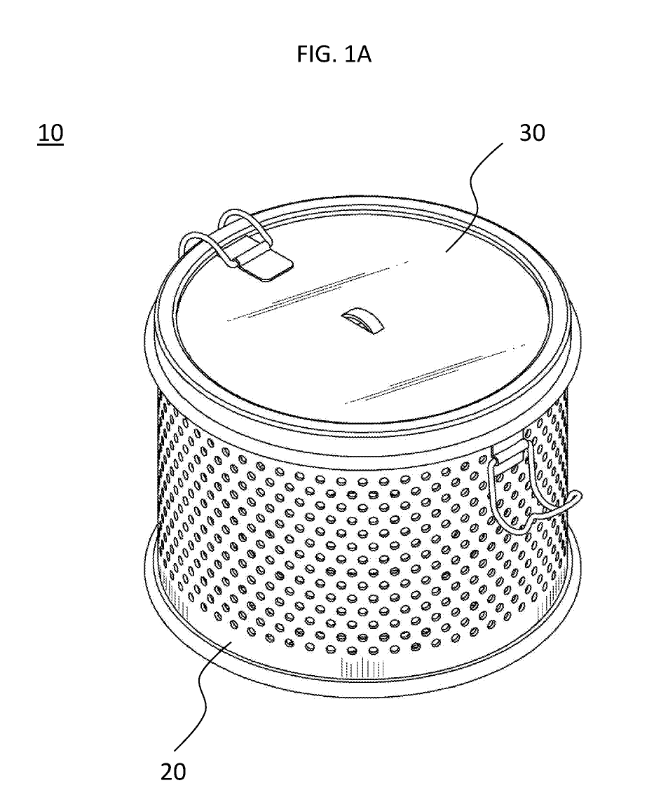

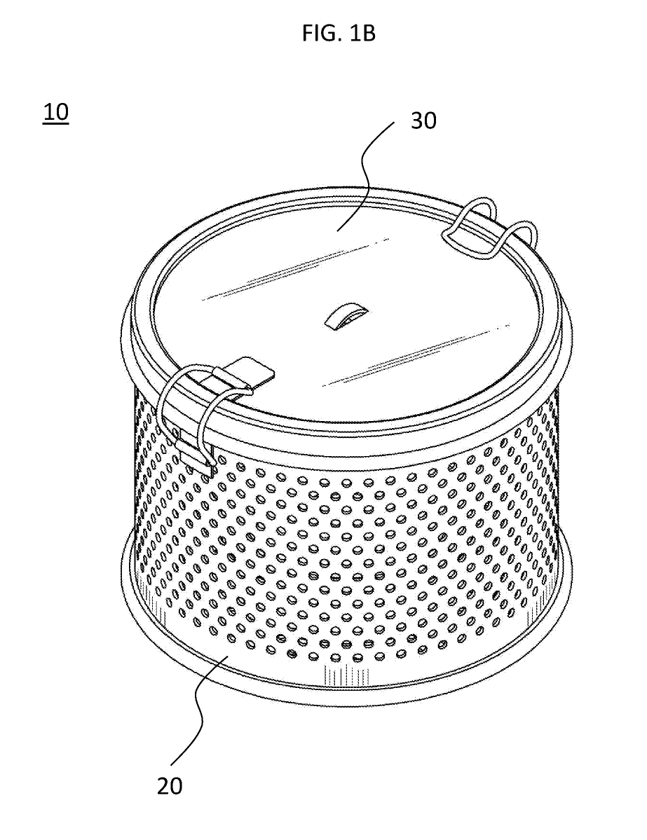

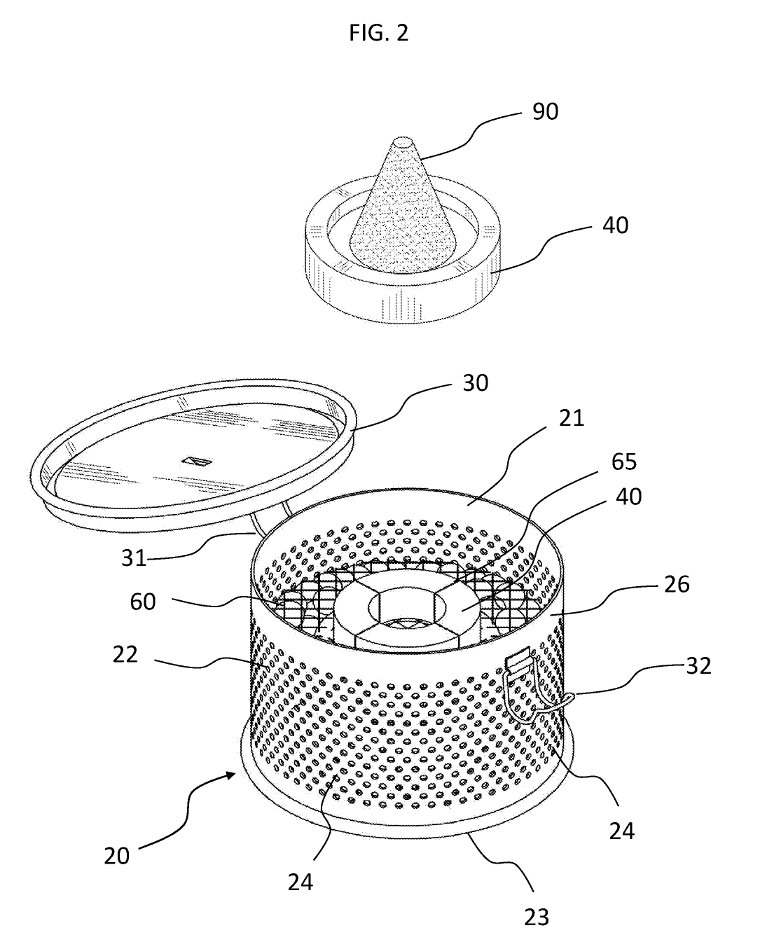

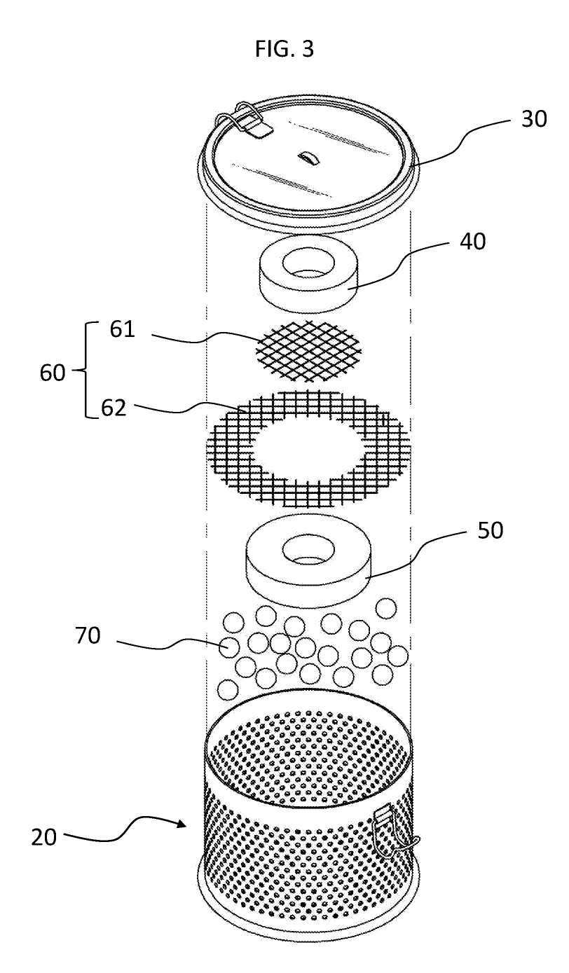

FIGS. 1A and 1B show front perspective views of a portable moxibustion therapy device according to one of embodiment of the present invention. FIG. 2 shows another perspective view of the portable moxibustion therapy device when a cover is opened along with moxa. FIG. 3 shows a front exploded view of the portable moxibustion therapy device.

The portable moxibustion therapy device 10 for treating pain of a body of user 100 using a burning moxa 90 made from dried mugwort, the portable moxibustion therapy device 10 including: a main body 20, having a top opening 21, a sidewall 22 and a bottom 23, and the sidewall 22 of the main body may includes a plurality of holes 24 on the sidewall 22 of the main body 20; a cover 30 for opening and closing the top opening 21 of the main body 20; a moxa container 40 which is substantially cylindrical and made of yellow soil in the main body 20; a cylindrical support 50 made of yellow soil placed the moxa container 40 blow in the main body 20; and a gauze 60 located between the moxa container 40 and the cylindrical support 50 in the main body 20. The moxa container 40 receives moxa therein and is placed in the main body 20.

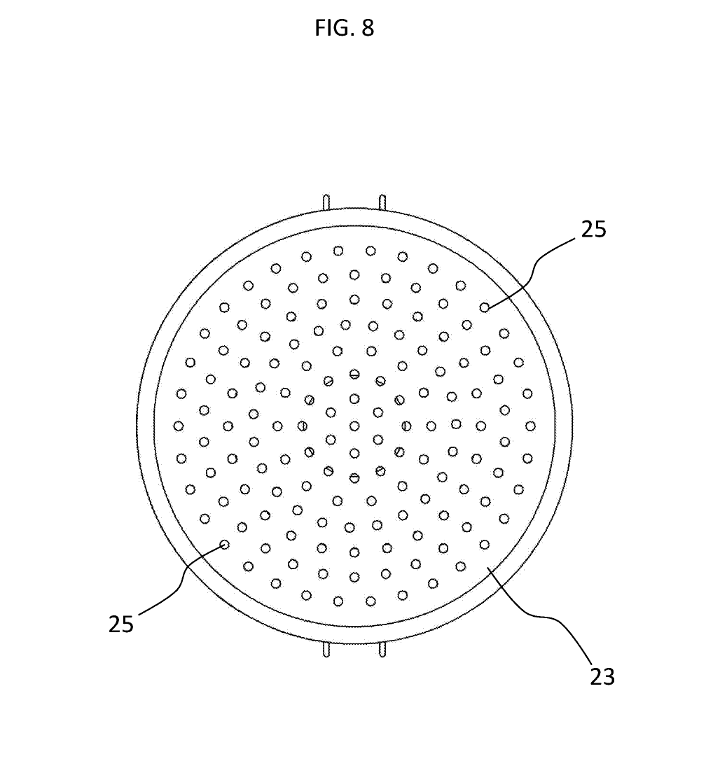

FIG. 8 shows a bottom plan view of the portable moxibustion therapy device.

The portable moxibustion therapy device further includes the plurality of holes 24 which is formed on the sidewall 22 of the main body 20 to supply air or oxygen to the moxa 90 and emit smoke of the moxa 90.

Preferably, the main body 20 may be a hollow cylindrical structure. The main body 20 and the cover 30 may be made of metal or stainless steel. The bottom 23 of the main body 20 may includes a plurality of holes 25 to supply oxygen to the moxa 90.

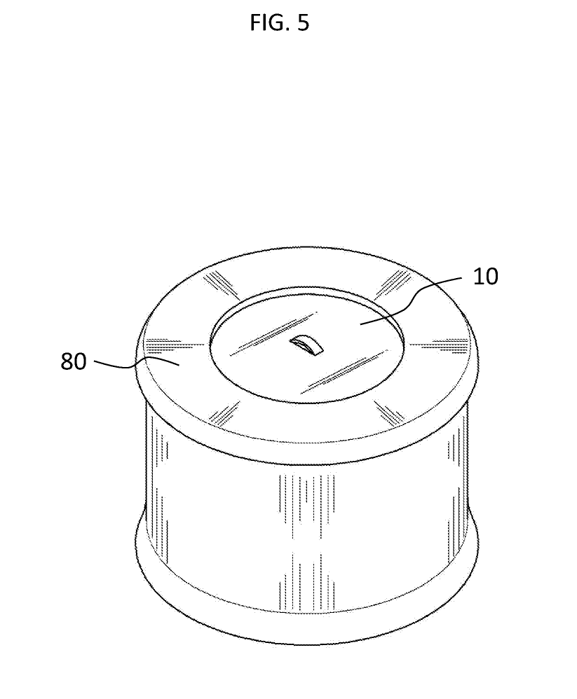

FIG. 5 shows another perspective view of the portable moxibustion therapy device which is wrapped or covered with fabric.

The portable moxibustion therapy device further includes a fabric 80 for reducing heat-transfer to the body of user 100. The fabric 80 may be substantially wrapped the sidewall 22 and the bottom 23 of the main body 20 and may be partially covered of the cover 30. The fabric material may be a cotton, a rayon, a regenerated cellulose fabric, a cotton-rayon blend, or a cotton-linen blend. The fabric 80 is an elastic fabric to snugly fit all around the main body 20.

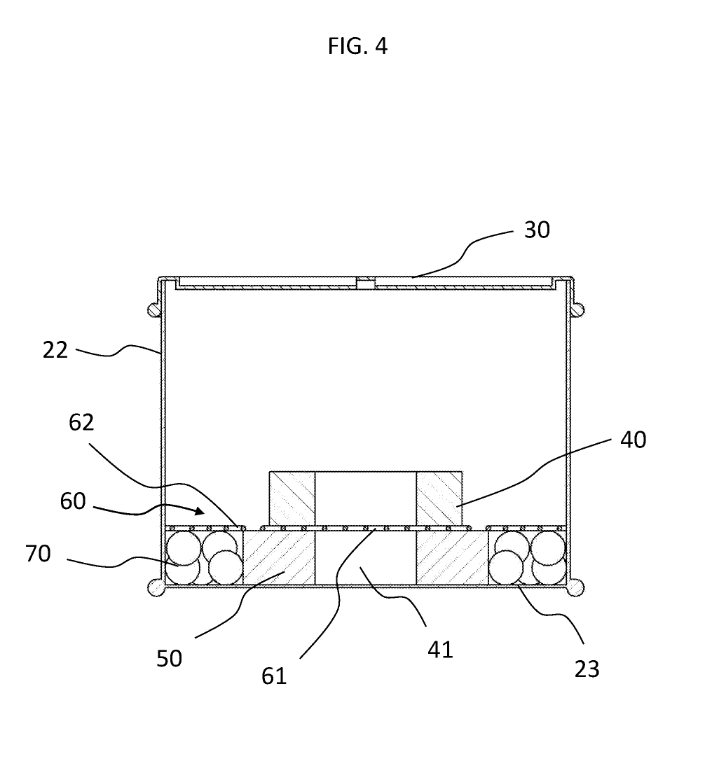

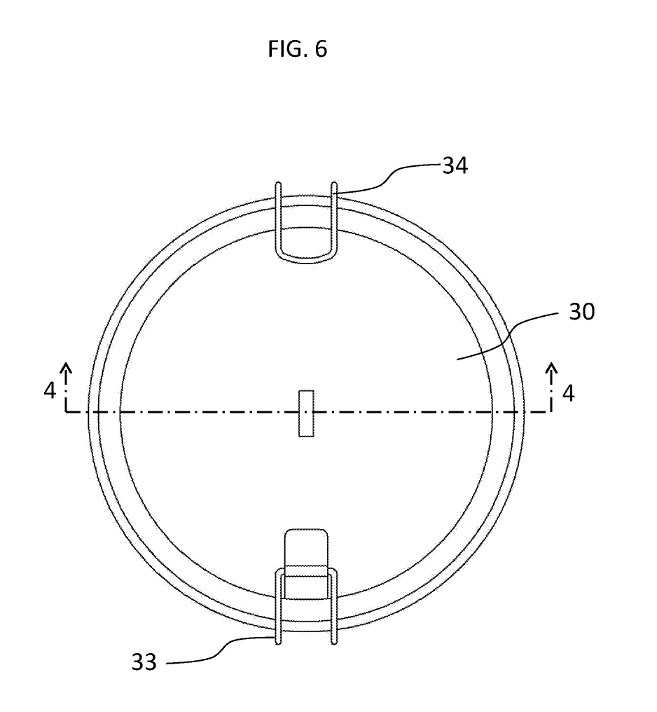

FIG. 4 shows a cross sectional view of the portable moxibustion therapy device along 4-4 line of FIG. 6. FIG. 6 shows a top plan view of the portable moxibustion therapy device according to the present invention.

The moxa container 40 and the cylindrical support 50 may form a cylindrical heat transferring passage 41 and inner diameters of the moxa container 40 and the cylindrical support 50 are substantially same. The portable moxibustion therapy device further includes a plurality of balls 70 made of mineral yellow soil or material. The plurality of balls 70 may be placed around the cylindrical support 40 and under the gauze 60. Preferably, the moxa container 40 which is substantially cylindrical, the gauze 60 and the cylindrical support 40 may be tied with metal wire 65 each other. Preferably, the gauze 60 securely fixed to the plurality of holes 24 of the sidewall 22. The gauze is a very fine wire mesh. A height of the cylindrical support 50 may be about 1.5 cm˜2.5 cm. In addition, a inner diameter of The moxa container 40 may be same or bigger than a bottom size of the moxa to receive the moxa.

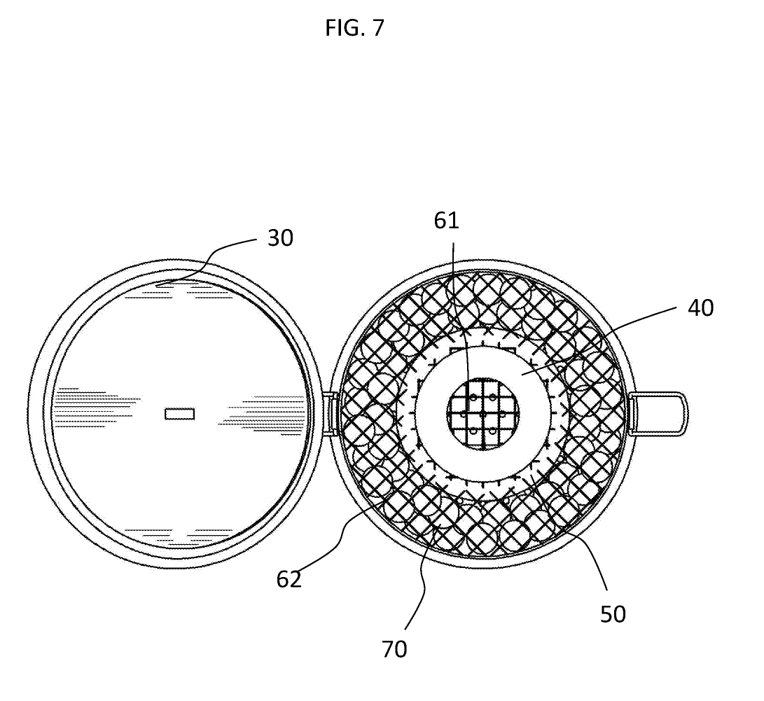

FIG. 7 shows a top plan view of the portable moxibustion therapy device when the cover is opened. The portable moxibustion therapy device further includes a first closing mean 31 and a second closing mean 32 fixedly attached on an upper portion 26 of the sidewall 22 of the main body 20. The first closing mean 31 may be a hinge 33 and the second closing mean 32 may be a hook 34.

As shown in FIGS. 2, 3 and 4, the portable moxibustion therapy device 10 for treating pain of a body of user 100 using a burning moxa 90 made from dried mugwort, the portable moxibustion therapy device 10 including a main body 20, having a top opening 21, a sidewall 22 and a bottom 23, and the sidewall 22 comprises a plurality of holes 24 on the sidewall 22 of the main body 20; a cover 30 for opening and closing the top opening 21 of the main body 20; a moxa container 40 which is substantially cylindrical and made of yellow soil in the main body 20; a cylindrical support 50 made of yellow soil placed the moxa container 40 blow in the main body 20; a plurality of balls 70 made of mineral material or yellow soil on the bottom of the main body; a first gauze 61 located between the moxa container 40 and the cylindrical support 50 in the main body 20; and a second gauze 62 covering the cylindrical support and the plurality of balls 70.

Preferably, the main body 20 may be a hollow cylindrical structure. The main body 20 and the cover 30 may be made of metal or stainless steel. The bottom 23 of the main body 20 may includes a plurality of holes 25 to supply air or oxygen to the moxa 90.

The portable moxibustion therapy device may further include a fabric 80 for reducing heat-transfer to the body of user 100. The fabric 80 may be substantially wrapped the sidewall 22 and the bottom 23 of the main body 20 and is partially covered of the cover 30. The moxa container 40 and the cylindrical support 50 may form a cylindrical heat transferring passage 41 and inner diameters of the moxa container 40 and the cylindrical support 50 may be substantially same.

The fabric 80 may be an elastic fabric to snugly fit all around the main body 20. Preferably, the fabric 80 may be substantially wrapped the sidewall 22 and the bottom 23 of the main body 20 and may be partially covered of the cover 30. The fabric material may be a cotton, a rayon, a regenerated cellulose fabric, a cotton-rayon blend, or a cotton-linen blend.

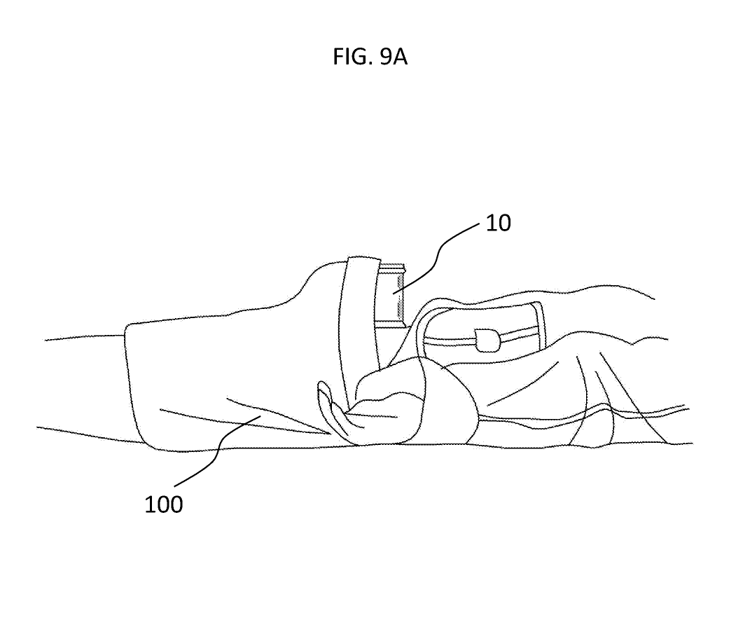

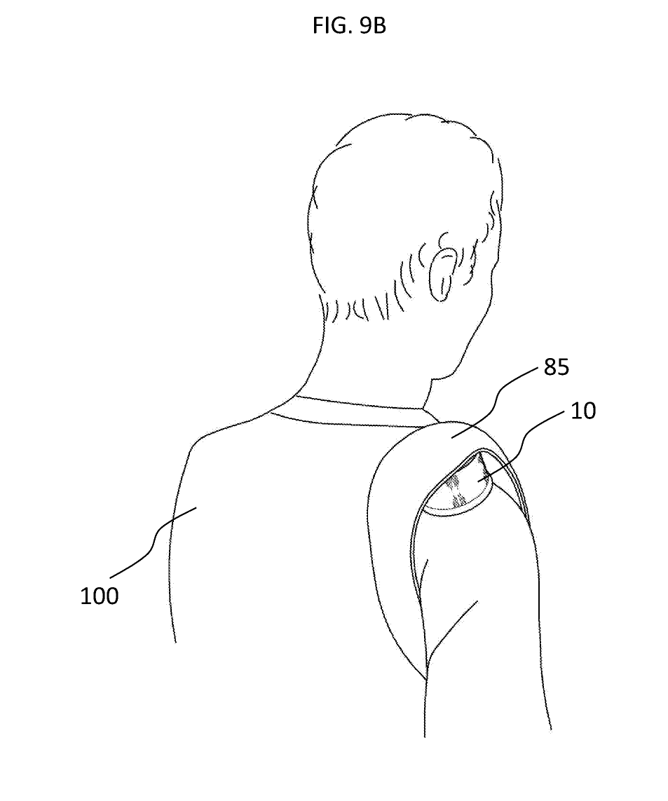

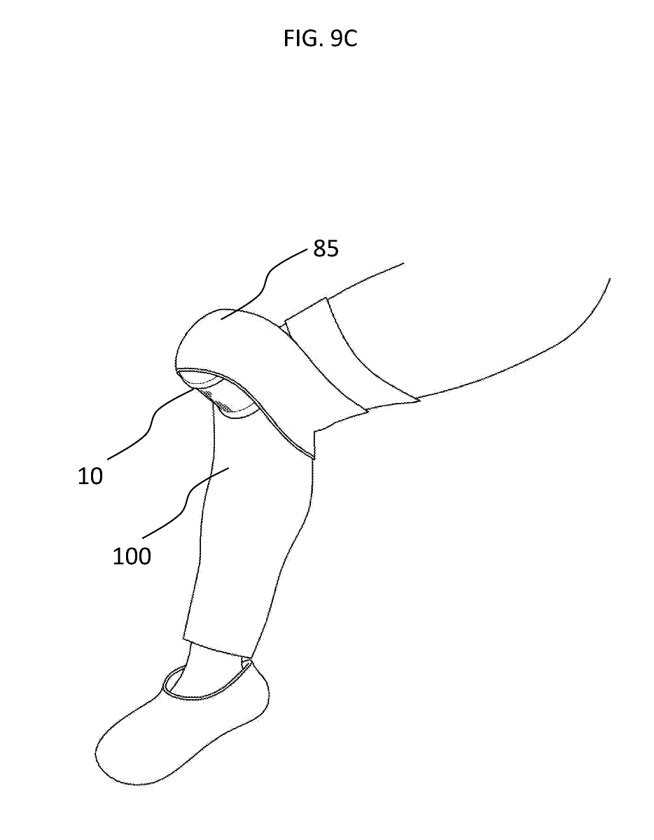

FIGS. 9A, 9B and 9C respectively show front elevation views of the portable moxibustion therapy device when the device attached the body of user such as back, shoulders and knees.

While the invention has been shown and described with reference to different embodiments thereof, it will be appreciated by those skilled in the art that variations in form, detail, compositions and operation may foe made without departing from the spirit and scope of the invention as defined by the accompanying claims.

What is claimed is:

1. A portable moxibustion therapy device (10) for relieving pain on a user's body (100) using a heat of combustion of moxa (90) made from dried mugwort, the portable moxibustion therapy device (10) comprising:
a main body (20), having a top opening (21), a sidewall (22) and a bottom (23), wherein the sidewall (22) comprises a plurality of holes (24);
a cover (30) for opening and closing the top opening (21) of the main body (20);
a moxa container (40) which is substantially cylindrical and made of yellow soil wherein the moxa container (40) receives moxa therein and is placed in the main body (20);
a cylindrical support (50) made of yellow soil for supporting the moxa container (40); and
a gauze (60) placed between the moxa container (40) and the cylindrical support (50).

2. The portable moxibustion therapy device of the claim 1, wherein the bottom (23) of the main body (20) further comprises a plurality of holes (25).

3. The portable moxibustion therapy device of the claim 1, further comprising a fabric (80) wherein the fabric (80) substantially wraps around the sidewall (22) and the bottom (23) of the main body (20) and partially covers the cover (30).

4. The portable moxibustion therapy device of the claim 1, wherein the moxa container (40) and the cylindrical support (50) form together a cylindrical heat transferring passage (41) and inner diameters of the moxa container (40) and the cylindrical support (50) are substantially same.

5. The portable moxibustion therapy device of the claim 1, further comprising a plurality of bails (70) made of mineral material or yellow soil.

6. The portable moxibustion therapy device of the claim 5, wherein the plurality of balls (70) is placed around the cylindrical support (40) below the gauze (60).

7. The portable moxibustion therapy device of the claim 1, wherein the moxa container (40), the gauze (60) and the cylindrical support (40) are tied together using a metal wire (65).

8. The portable moxibustion therapy device of the claim 7, wherein the gauze (60) is fixed to the plurality of holes (24) of the sidewall (22).

9. The portable moxibustion therapy device of the claim 7, wherein a height of the cylindrical support (50) is about 1.5 cm˜2.5 cm.

10. The portable moxibustion therapy device of the claim 1, further comprising a first closing mean (31) and a second closing mean (32) fixedly attached to an upper portion (26) of the sidewall (22) of the main body (20).

11. The portable moxibustion therapy device of the claim 10, wherein the first closing mean (31) is a hinge (33) and the second closing mean (32) is a hook (34).

12. The portable moxibustion therapy device of the claim 1, wherein, the main body (20) is constructed to form a hollow cylinder.

13. The portable moxibustion therapy device of the claim 1, wherein the main body (20) and the cover (30) are made of metal or stainless steel.

14. The portable moxibustion therapy device of the claim 3, wherein the fabric (80) is an elastic fabric to snugly fit all around the main body (20).

15. A portable moxibustion therapy device (10) for relieving pain on a user's body (100) using a heat of combustion of moxa (90) made from dried mugwort, the portable moxibustion therapy device (10) comprising:
a main body (20), having a top opening (21), a sidewall (22) and a bottom (23), wherein the sidewall (22) comprises a plurality of holes (24);
a cover (30) for opening and closing the top opening (21) of the main body (20);
a moxa container (40) which is substantially cylindrical and made of yellow soil wherein the moxa container (40) is placed in the main body (20);
a cylindrical support (50) made of yellow soil for supporting the moxa container (40);
a plurality of balls (70) made of mineral material or yellow soil placed on the bottom of the main body (20);
a first gauze (61) located between the moxa container 40 and the cylindrical support (50); and
a second gauze (62) placed on or above the cylindrical support (50) and the plurality of balls (70).

16. The portable moxibustion therapy device of the claim 15, wherein the bottom (23) of the main body (20) further comprises a plurality of holes (25).

17. The portable moxibustion therapy device of the claim 15, further comprising a fabric (80) wherein the fabric (80) substantially wraps around the sidewall (22) and the bottom (23) of the main body (20) and partially covers the cover (30).

18. The portable moxibustion therapy device of the claim 15, wherein the moxa container (40) and the cylindrical support (50) form together a cylindrical heat transferring passage (41) and inner diameters of the moxa container (40) and the cylindrical support (50) are substantially same.

19. The portable moxibustion therapy device of the claim 15, wherein the main body (20) is constructed to form a hollow cylinder and wherein the main body (20) and the cover (30) are made of metal or stainless steel.

20. The portable moxibustion therapy device of the claim 17, wherein the fabric (80) is an elastic fabric to snugly fit all around the main body (20).
